(19) United States
(12) Patent Application Publication
(10) Pub. No.: US 2021/0106767 A1
(43) Pub. Date: Apr. 15, 2021

(54) HYPODERMIC NEEDLE PROTECTIVE COVER

(71) Applicant: TRICENTUM TECHNOLOGIES LLC, ST. LOUIS, MO (US)

(72) Inventors: SHARON SABO, ST. LOUIS, MO (US); CIANAN SABO, ST. LOUIS, MO (US)

(21) Appl. No.: 17/068,577

(22) Filed: Oct. 12, 2020

Related U.S. Application Data
(60) Provisional application No. 62/913,247, filed on Oct. 10, 2019.

(51) Int. Cl. A61M 5/32; A61M 5/31; A61M 5/50

(52) U.S. Cl. CPC A61M 5/3202; A61M 5/50; A61M 5/3129

(57) ABSTRACT

Described herein is a needle protective cover having a cylindrical barrel, a funnel, and a support mechanism. The cylindrical barrel has a proximal end and a distal end and is operable to receive a hypodermic needle or hypodermic needle sheath. The funnel is at the proximal end of the cylindrical barrel. The support mechanism surrounds the cylindrical barrel and funnel, such that the support mechanism is operable to prevent the needle protective cover from rolling on a surface and increases a distance from the funnel to the surface. The support mechanism may be a triangular body and/or one or more fins.

### CROSS REFERENCE TO RELATED APPLICATIONS

This application claims priority to U.S. Provisional Application No. 62/913,247, filed Oct. 10, 2019, the contents of which are incorporated by reference in its entirety.

### FIELD

The invention relates generally to a medical device for use as a protective sheath over a hypodermic needle.

### BACKGROUND

Every year 13 million people in the United States engage in self-management of a medical condition involving self-injection of a medication. There are a significant number of these patients which also suffer a condition resulting in reduced dexterity or handgrip strength, have tremors, have fear of inadvertent injury, and/or have diminished ability to learn proper technique for insulin injection. Many patients who self-inject use disposable hypodermic needles with proactive sheaths. In this patient population, unsheathing and resheathing the needle can cause an increased risk of needle stick injuries (NSIs) and/or contamination of the tip.

As presented herein, a hypodermic needle protective cover has been developed to reduce the risk of NSIs and/or contamination of the needle tip.

### BRIEF SUMMARY

Provided herein is a needle protective cover. The needle protective cover may include a cylindrical barrel having a proximal end and a distal end, a funnel at the proximal end of the cylindrical barrel, and a support mechanism surrounding the cylindrical barrel and funnel. The cylindrical barrel and the funnel are operable to receive a hypodermic needle or hypodermic needle sheath. The support mechanism is operable to prevent the needle protective cover from rolling on a surface and increases a distance from the funnel to the surface.

The support mechanism may have a triangular body and/or one or more fins. In some aspects, the fins are evenly spaced around the cylindrical barrel. In an aspect, two or more fins may be connected together to form a hollow triangular portion. The distal end of the fins may be flat or extended beyond the cylindrical barrel and drawn to a point. In other aspects, the cylindrical barrel is extended beyond the fins to a point. The one or more fins may include one or more openings. The one or more openings may be circular, oval, or rectangular. The one or more fins may have a flat edge, a pointed edge, a rounded edge, a scalloped edge, or serrated edge.

The needle protective cover may further include a cylinder operable to hold a light emitter. The cylinder may be connected to and parallel to the cylindrical barrel or the cylinder may be within the support mechanism. The light emitter may be a chemical light stick operable to emit in the visible light or UV portion of the spectrum.

The needle protective cover may further include a visualization marker. The visualization marker may include metal or iodine loaded nanoparticles, a metal coil, and/or a pigment imbedded in the needle protective cover.

The proximal end of the funnel may be flat, scalloped, or rounded. The funnel may further include a Luer Lock hub.

In some aspects, the needle protective cover is pre-sterilized prior to packaging.

The advantages and the features of the present invention will become better understood with reference to the following and the more detailed description and the claims taken in conjunction with the accompanying drawings.

### BRIEF DESCRIPTION OF THE DRAWINGS

Implementations of the present technology will now be described, by way of example only, with reference to the attached figures, wherein:

FIG. 1A is a top view of the needle protective cover with two fins, in one example;

FIG. 1B is a side view of the needle protective cover with two fins, in one example;

FIG. 2A is a top view of the needle protective cover with two fins, in one example;

FIG. 2B is a side view of the needle protective cover with two fins, in one example;

FIG. 3A is a top view of the needle protective cover with three fins, in one example;

FIG. 3B is a side view of the needle protective cover with three fins, in one example;

FIG. 4A is a top view of the needle protective cover with four fins, in one example;

FIG. 4B is a side view of the needle protective cover with four fins, in one example;

FIG. 5A is a top view of the needle protective cover with five fins, in one example;

FIG. 5B is a side view of the needle protective cover with two fins, in one example;

FIG. 6A is a top view of the needle protective cover with three fins, in one example;

FIG. 6B is a side view of the needle protective cover with three fins, in one example;

FIG. 7A is a top view of the needle protective cover with a triangular body, in one example;

FIG. 7B is a side view of the needle protective cover with a triangular body, in one example;

FIG. 8A is a top view of the needle protective cover with three fins, in one example;

FIG. 8B is a side view of the needle protective cover three two fins, in one example;

FIG. 9A is a top view of the needle protective cover with three fins, in one example;

FIG. 9B is a side view of the needle protective cover with three fins, in one example;

FIG. 10A is a top view of the needle protective cover with a triangle feature and one fin, in one example;

FIG. 10B is a side view of the needle protective cover with a triangle feature and one fin, in one example;

FIG. 11A is a top view of the needle protective cover with a triangular body, in one example;

FIG. 11B is a side view of the needle protective cover with a triangular body, in one example;

FIG. 12A is a top view of the needle protective cover with a triangular body, in one example;

FIG. 12B is a side view of the needle protective cover with a triangular body, in one example;

FIG. 13A is a top view of the needle protective cover with three fins, in one example;

FIG. 13B is a side view of the needle protective cover with three fins, in one example;

FIG. 14A is a top view of the needle protective cover with three fins, in one example;

FIG. 14B is a side view of the needle protective cover with three fins, in one example;

FIG. 15A is a top view of the needle protective cover with three fins, in one example;

FIG. 15B is a side view of the needle protective cover with three fins, in one example;

FIG. 16A is a top view of the needle protective cover with three fins, in one example;

FIG. 16B is a side view of the needle protective cover with three fins, in one example;

FIG. 17A is a top view of the needle protective cover with three fins, in one example;

FIG. 17B is a side view of the needle protective cover with three fins, in one example;

FIG. 18A is a top view of the needle protective cover with three fins, in one example;

FIG. 18B is a side view of the needle protective cover with three fins, in one example;

FIG. 19A is a top view of the needle protective cover with three fins, in one example;

FIG. 19B is a side view of the needle protective cover with three fins, in one example;

FIG. 20A is a top view of the needle protective cover with three fins, in one example;

FIG. 20B is a side view of the needle protective cover with three fins, in one example;

FIG. 21 is a side view of the needle protective cover with three fins, in one example;

FIG. 22A is a top view of the needle protective cover with three fins, in one example;

FIG. 22B is a side view of the needle protective cover with three fins, in one example;

FIG. 23A is a top view of the needle protective cover with the fins extended and drawn to a point, in one example;

FIG. 23B is a side view of the needle protective cover with the fins extended and drawn to a point, in one example;

FIG. 24A is a top view of the needle protective cover with the cylinder extended to a point, in one example;

FIG. 24B is a side view of the needle protective cover with the cylinder extended to a point, in one example;

FIG. 25A is a top view of the needle protective cover with a flat funnel, in one example;

FIG. 25B is a side view of the needle protective cover with a flat funnel, in one example;

FIG. 26A is a top view of the needle protective cover with a scalloped funnel, in one example;

FIG. 26B is a side view of the needle protective cover with a scalloped funnel, in one example;

FIG. 27A is a top view of the needle protective cover with a circular funnel, in one example;

FIG. 27B is a side view of the needle protective cover with a circular funnel, in one example;

FIG. 28A is a top view of the needle protective cover with a Luer Lock, in one example; and

FIG. 28B is a side view of the needle protective cover with a Luer Lock, in one example.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented. The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, “substantially cylindrical” means that the object resembles a cylinder, but can have one or more deviations from a true cylinder. The terms “comprising,” “including” and “having” are used interchangeably in this disclosure. The terms “comprising,” “including” and “having” mean to include, but not necessarily be limited to the things so described.

Recent changes in needle protective sheath design has focused reducing needle sticks with covers that can be actuated one-handed to recover the needle after a single use. While these are most appropriate is some cases, the standard hypodermic needles with removable sheaths remain the most common in the clinical environment. The sheath needs to be recapped after use for proper disposal of the needle. In addition, patients who self-inject may draw up multiple doses from a single vial and recap with the intent to dose later.

To recap the needle properly, the sheath is placed on a surface and the needle is placed into the sheath using only one hand. Most designs incorporate a design feature (knurled area, squared edges, or nubs) to prevent easy rolling of the sheath when placed on a flat surface but these are insufficient for individuals with tremor. The closeness of the center of the protective sheath to the working surface they rest upon make a challenging recapping target for a patient with reduced dexterity or tremor. The narrow diameter of the sheath can be difficult for the arthritic patient to easily manipulate when removing from its protective packaging therefore increasing the likelihood of NSI.

Home health trainers emphasize proper single-handed needle technique, however difficulties over using current protective sheaths leads to frustration , culminating in poor handling technique, increased risk of NSI and/or needle tip contamination. This may also lead to non-compliance with medication regimen.

Provided herein is a needle protective cover for patients with limited dexterity and/or impaired motor control who must self-inject medications. For example, the needle protective cover may improve the resheathing of a hypodermic needle by reducing rolling of the cover and increasing the distance from the surface to the barrel of the cover. The needle protective cover may help a self-injecting patient reduce the risk of NSI and/or needle tip contamination. The needle protective cover is intended to streamline within current workflows that self-injecting patients follow.

As seen in the figures, the needle protective cover 100 has a substantially cylindrical barrel 102 surrounded by a support mechanism 104 to increase the distance between the barrel and a surface when the needle protective cover is placed longitudinally on the surface. The needle protective cover 100 may further include a funnel 114 connected to the proximal end of the cylindrical barrel 102 which also may be surrounded by the support mechanism 104. In some examples, the needle protective cover may have a length of about 0.5 inches to about 10 inches.

The support mechanism prevents the needle protective cover from rolling and raises the opening of the cylindrical barrel above the unsterile surface. In some examples, the support mechanism may form a generally triangular shape around the cylindrical barrel, giving the needle protective cover a substantially triangular cross-section. For example, the support mechanism may be formed from a triangular body and/or one or more fins. Triangle-derived designs increase the distance between the needle protective lumen and the resting surface when the sides of the triangular body/fins are increased. This increase in height reduces the chance of shoveling (touching the needle tip to the working surface prior to engaging the protective lumen) where the needle tip can become potentially contaminated. Enlargement of dimensions also increases the overall volume and surface area of the device making it easier to manipulate by individuals with decreased fine motor skills.

The support mechanism may extend the length or a portion of the length of the cylindrical barrel and funnel. For example, the length of the fins and/or triangular body may be the entire length of the cylindrical barrel. In other examples, the length of the fins and/or triangular body may be less than the length of the cylindrical barrel. In additional examples, the length of the fins and/or triangular body may be more than the length of the cylindrical barrel. In some examples, the support mechanism may have a length of about 0.5 inches to about 10 inches. The fins and/or triangular body may have a length of about 0.5 in to 2 in, 1 in to 3 in, 2 in to 4 in, 3 in to 5 in, 4 in to 6 in, 5 in to 7 in, 6 in to 8 in, 7 in to 9 in, or 8 in to 10 in.

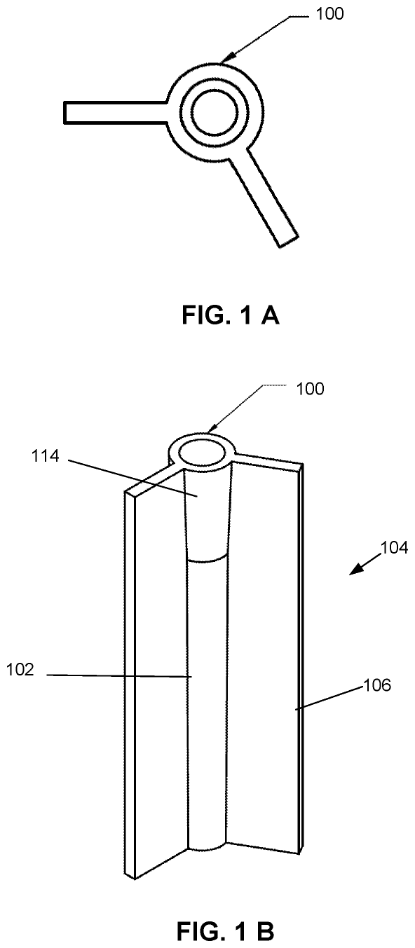

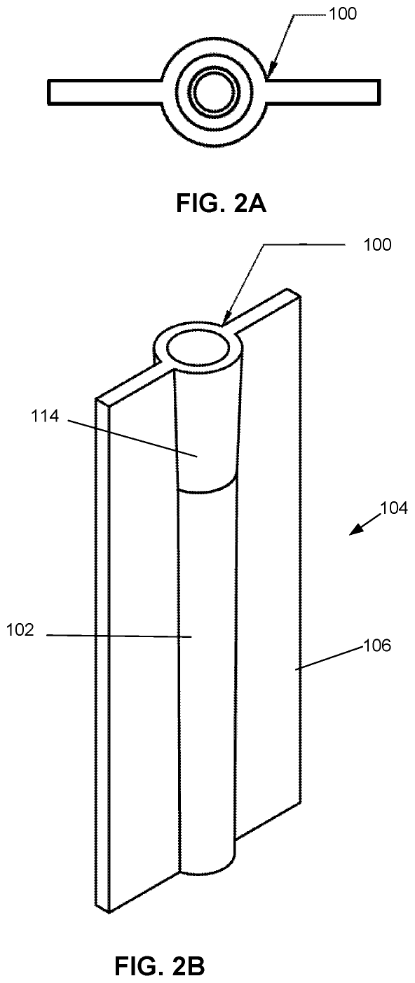

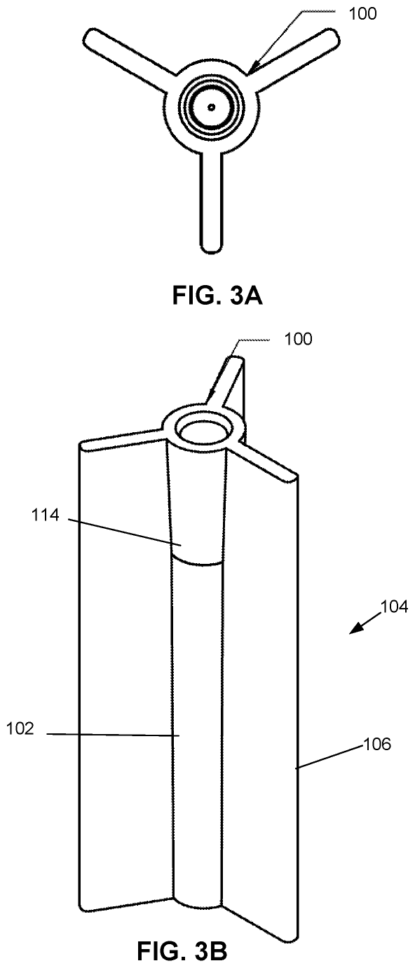

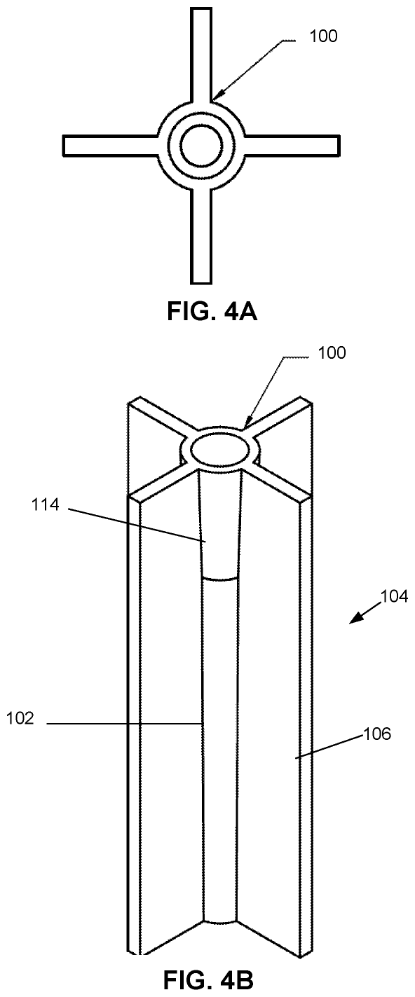

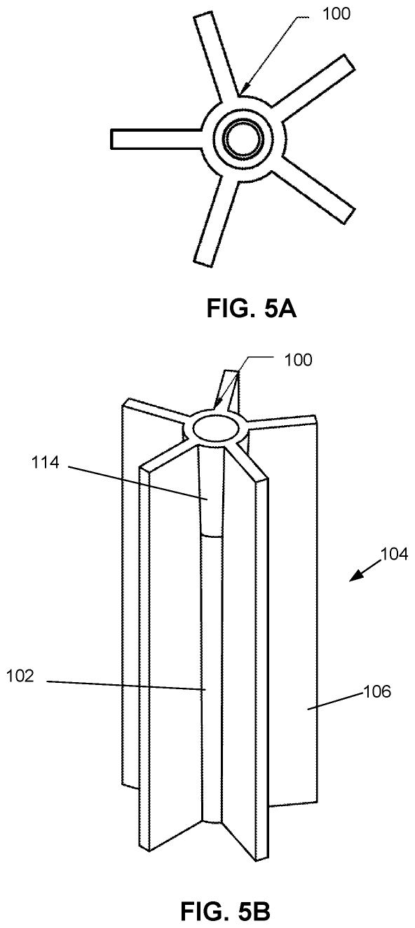

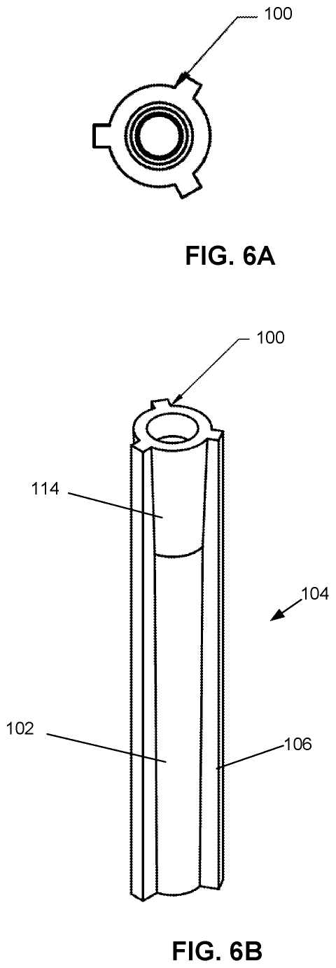

The support mechanism may include plurality of fins spaced around the barrel. The number of fins may vary between two and five. In some examples, the support mechanism does not have a generally triangular shape but still increases the height and surface area of the needle protective cover, as seen in FIGS. 1A-2B. In this example, the support mechanism 104 includes two fins 106 in various arrangements around the circumference of the barrel 102. The fins 106 may either be parallel (FIGS. 2A-2B) or offset (FIGS. 1A-1B). In some examples, the needle protective cover may have three fins, as seen in FIGS. 3A-3B. The fins 106 may be evenly spaced around the barrel to provide a generally triangular shape. In another example, the support mechanism 104 includes four fins 106 around the circumference of the barrel 102, as seen in FIGS. 4A-4B. In yet another example, the support mechanism 104 includes five fins 106 around the circumference of the barrel 102, as seen in FIGS. 5A-5B.

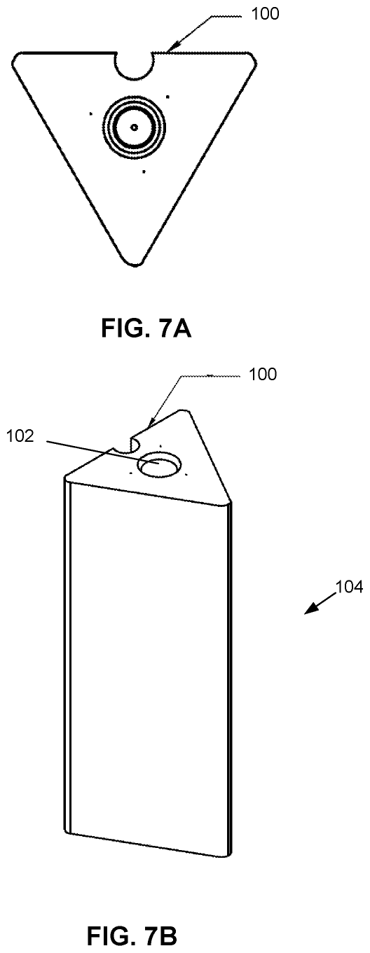

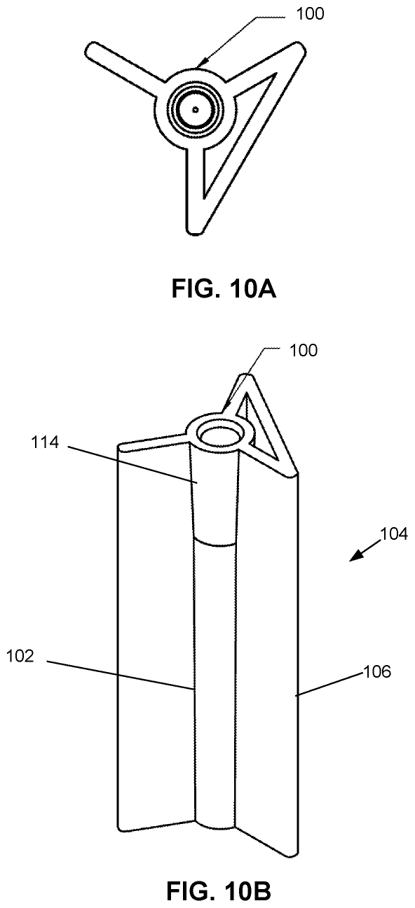

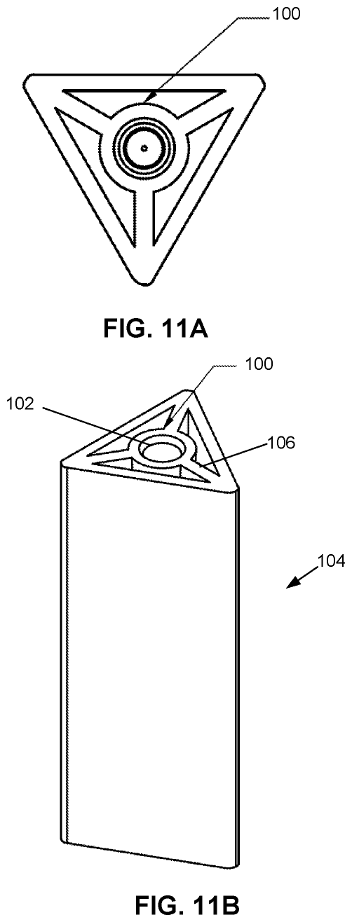

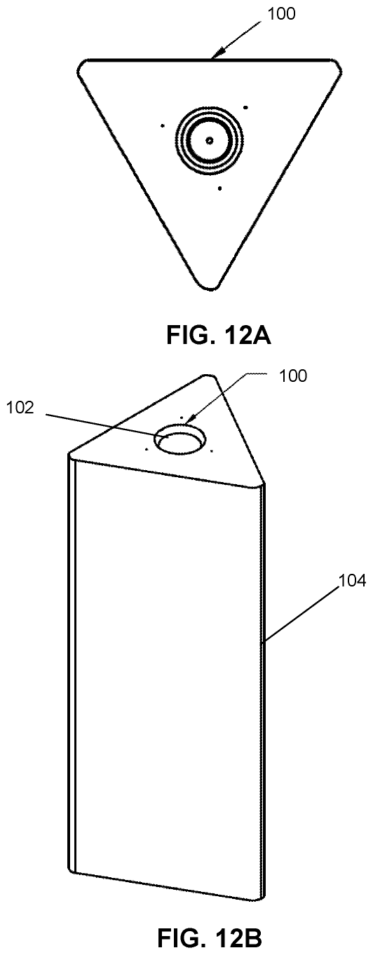

In other examples, the support mechanism may be a filled triangular body, as seen in FIGS. 7A-7B and 12A-12B, a hollow triangular body, as seen in FIGS. 11A-11B, or a hybrid having a triangular portion and single fin, as seen in FIGS. 10A-10B. Alternatively, one or more of the fins may be connected to an adjacent fin to provide a flat side, as shown in FIGS. 10A-11B. The volume between the fins may be either hollow or filled as shown in FIGS. 11A-12B.

The fins, connected fins, and/or side of a triangular body may form a flat side 108. This flat side 108 may provide space for a written label indicating what medication is involved with the needle, a barcode containing the same information in digital format, a magnetic strip for attaching to ferromagnetic surfaces, and/or a Velcro strip for attaching to matching Velcro surface. The addition of a smooth, flat surface on one or more sides of the needle protective cover is an added benefit not directly related to handling characteristics. This surface offers the opportunity to be written on with an indelible marker or have a bar code applied via HCP or Pharmacy as needed—a feature not available in existing protective covers. In some examples, the support mechanism 104 may further include a notch, as seen in FIGS. 7A-7B, which may hold a solid colored rod for medication type identification.

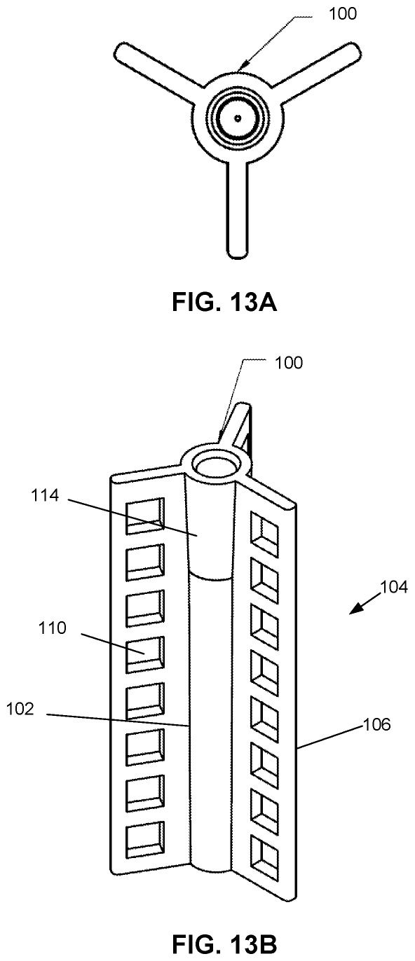

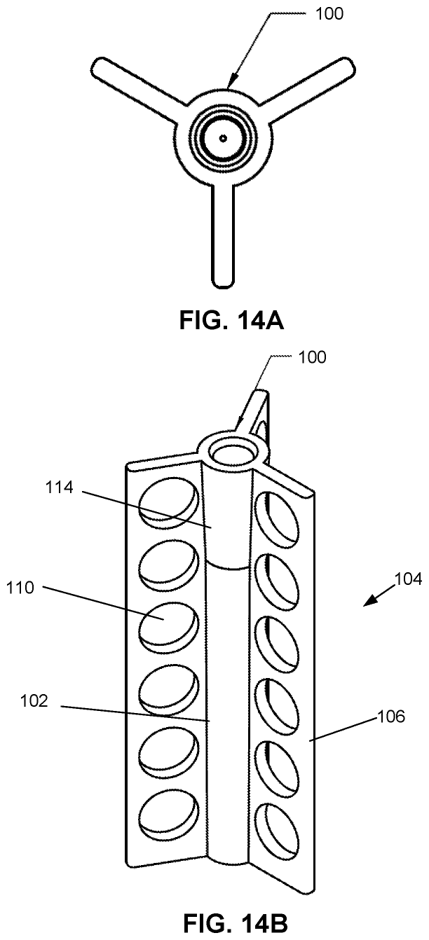

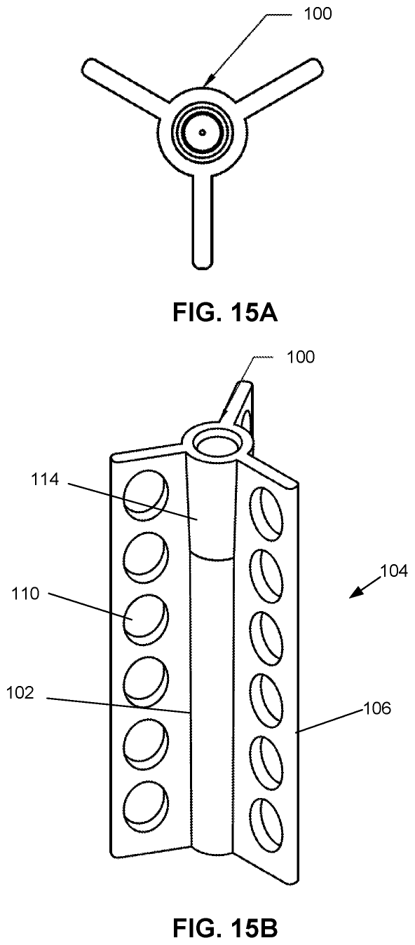

The fins 106 may have one or more openings 110 along the length of the fin. The openings may be circles, ovals, or squares, as shown in FIGS. 13B, 14B, and 15B. In various examples, each fin may have 1, 2, 3, 4, 5, 6, 7, 8, 9, or 10 openings. The openings may be the same or different sizes. The openings may reduce the amount of material used when forming the needle protective cover. In addition, the openings may reduce the weight of the needle protective cover.

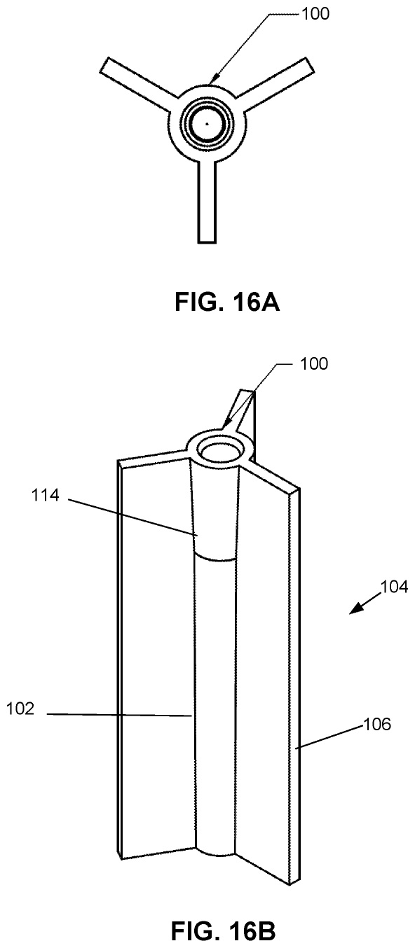

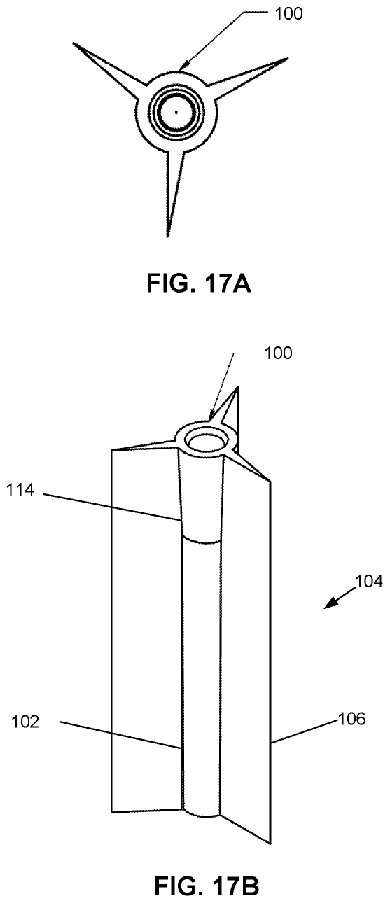

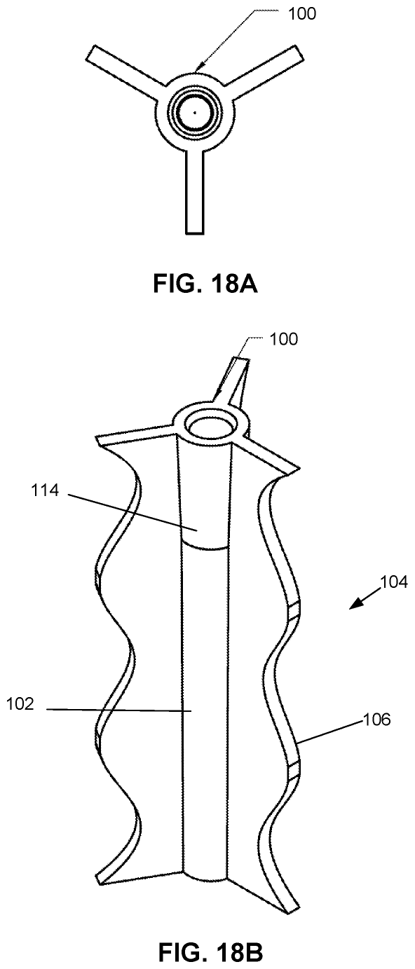

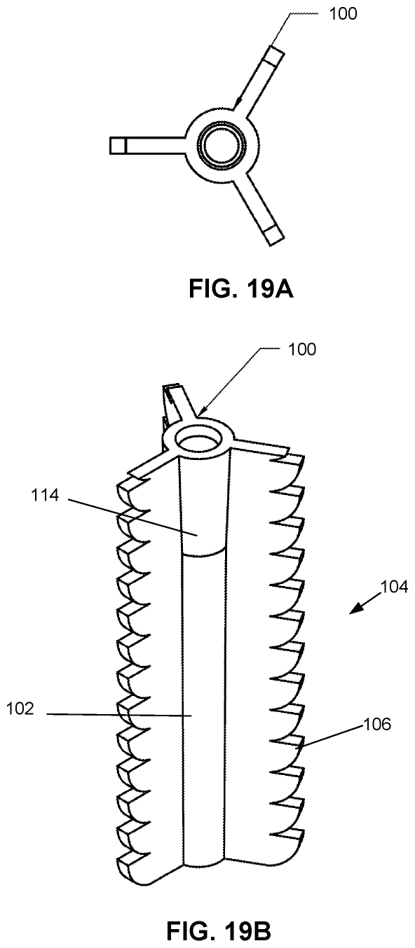

In some examples, the edges of the fins may be either be squared, as seen in FIGS. 16A-16B, or pointed, as shown in FIGS. 17A-17B for the purposes of increasing the ability to grip the needle protective cover. In other examples, the edges may also be rounded as shown in FIGS. 3A-3B for the purposes of comfort. In additional examples, the fin edges may have a scalloped form as shown in FIG. 18B for the purpose of increasing finger grip. In another example, the fin edges may be serrated, similar to direction denticles with one rounded edge and one square edge, as shown in FIG. 19B for the purposes of creating increase friction when pulling the device in one direction.

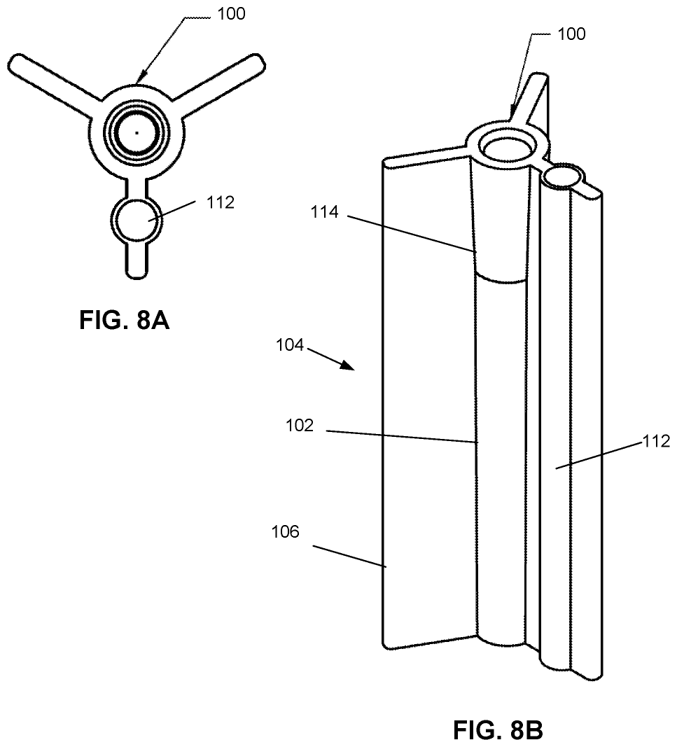

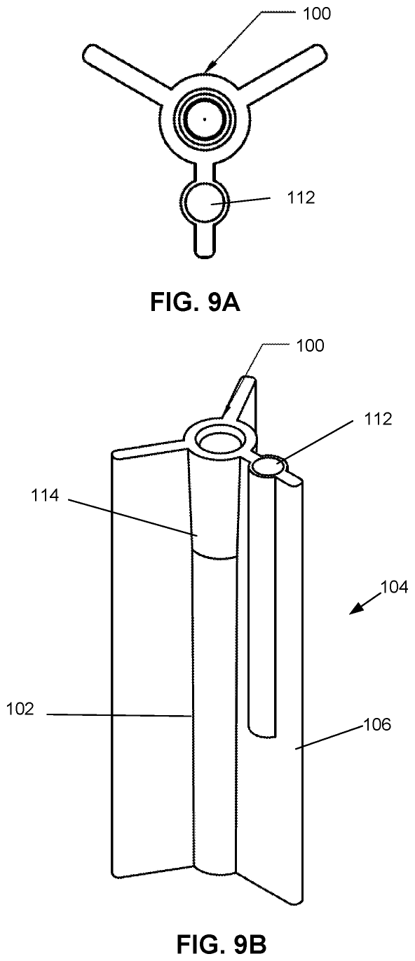

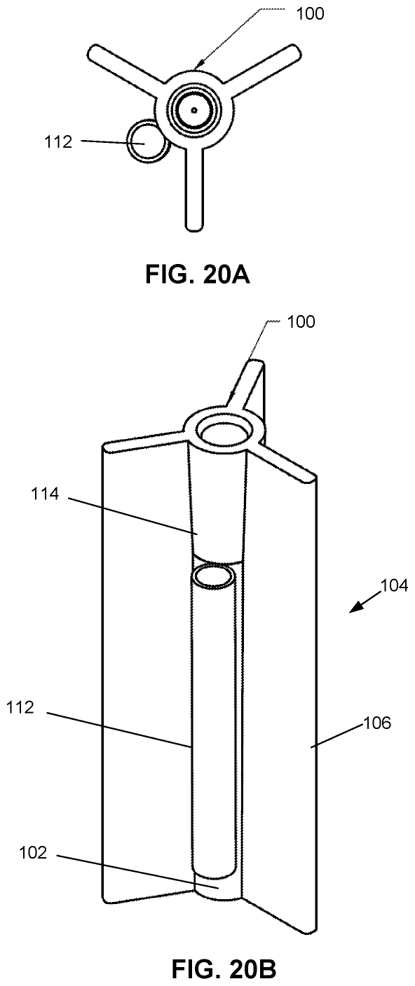

The needle protective cover 100 may further include a second cylinder 112 that may run adjacent and parallel to the cylindrical barrel. The second cylinder may be operable to hold a light emitter. For example, the second cylinder may hold a chemical light stick that emits either in the visible light or UV portion of the spectrum. In an example, the second cylinder may be connected and adjacent to the cylindrical barrel, as shown in FIGS. 20A-20B. In another example, the second cylinder may be built into a fin as shown in FIGS. 8A-8B. When in a fin, the second cylinder may be shortened to less than the length of the fin, as shown in FIGS. 9A-9B. Alternatively, a triangular body may include a notch as seen in FIGS. 7A-7B, which may hold a chemical light stick that emits either in the visible light or UV portion of the spectrum.

The material of the needle protective cover may be one of many polymers common in the medical device field. Non-limiting examples of polymers include polypropylene, polyethylene, and acrylonitrile butadiene styrene. Additionally, the needle protective cover may be made from one of many starch or cellulose based biopolymers that are biodegradable or that can be incinerated without producing harmful organic byproducts. The material used to create the needle protective cover may be extruded, thermoset in molds, or 3D printed. If the device is 3D printed, two extruder heads may be used to create black and white layers in the form of a barcode.

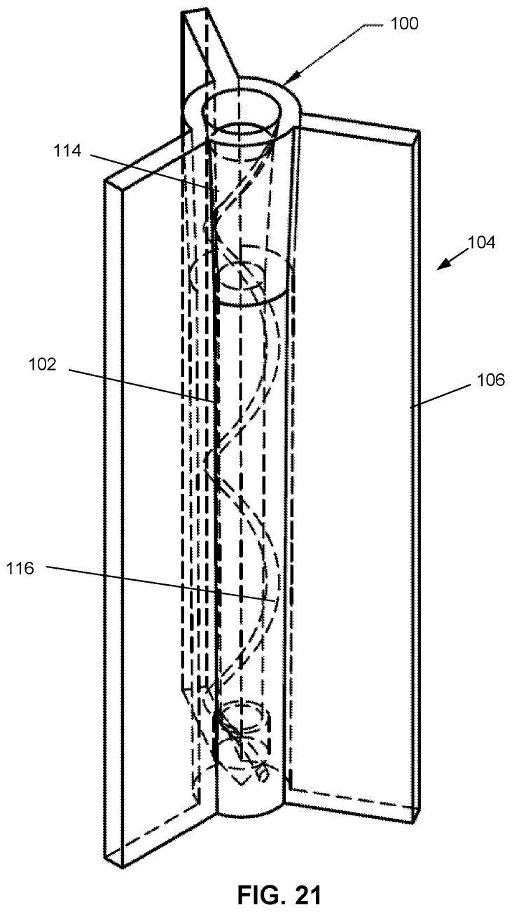

The needle protective cover may further include a visualization marker to more easily view the device when in a medical x-ray fluoroscopy field. In some examples, metal or iodine loaded nanoparticles may be included in the polymer material of the needle protective cover. In other examples, a metal coil 116 made of platinum, or similar, may be embedded in the barrel lumen as shown in FIG. 21. In some examples, pigments may be added to the polymer material. Theses pigment may be either for color in visible light, phosphorescent, or emissive when exposed to UV light.

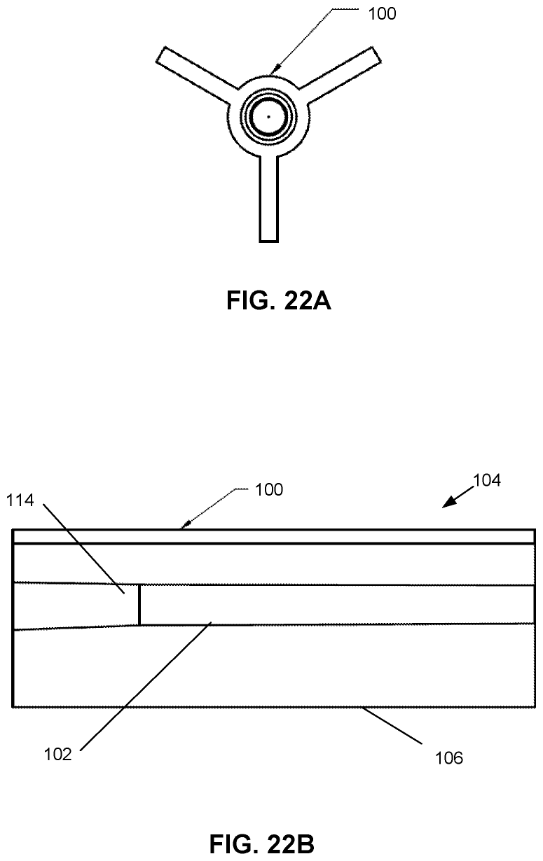

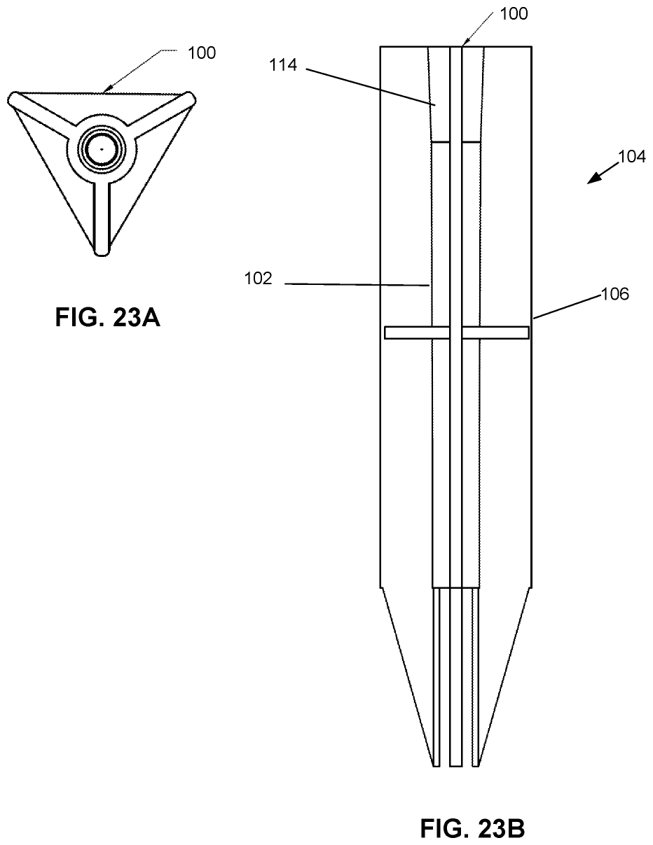

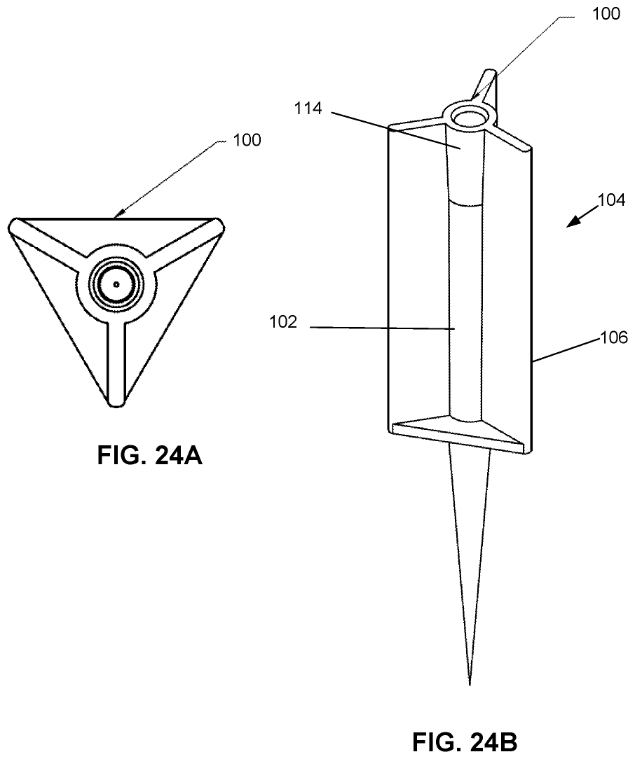

The distal end of the needle protective cover may be a closed. In various examples, the distal end may be flat as shown in FIG. 22B, the fins extended and drawn to a point as shown in FIG. 23B, or the cylinder extended to a point, or spike, as shown in FIG. 24B. In the case of the pointed end designs, the purpose of the feature is to allow the needle protective cover to be partially driven into soft soil or sand. For example, in the case of field medicine (Department of Defense, Civilian Emergency Response Teams, and Emergency Medical Responders) there may not be a sterile surface to work from, so the needle protective cover may be stuck into the soil, holding the sterile opening above ground level.

The cylindrical barrel is of sufficient length and width to cover a hypodermic needle. For example, the length of the cylindrical barrel may range from about 0.5 in to about 2 in, 1 in to 3 in, 2 in to 4 in, 3 in to 5 in, 4 in to 6 in, 5 in to 7 in, 6 in to 8 in, 7 in to 9 in, or 8 in to 10 in. In additional examples, the width of the cylindrical barrel may range from about 0.05 in to 0.1 in, 0.1 in to 0.2 in, 0.2 in to 0.3 in, or 0.3 in to 0.4 in. The cylindrical barrel may be sized on the smaller end to replace a current needle protective sheath, or the barrel diameter may be increased such that it fits over the current family of needle protective sheaths.

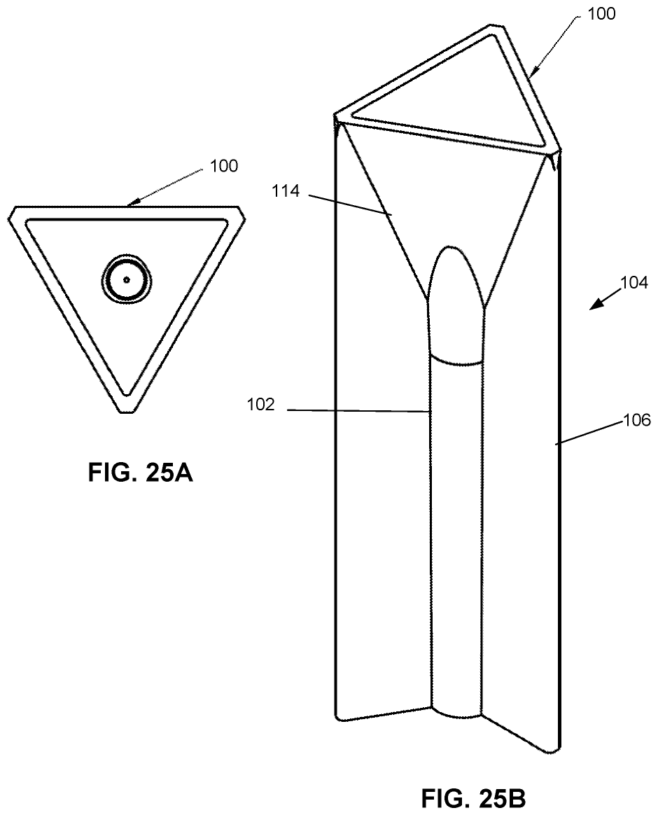

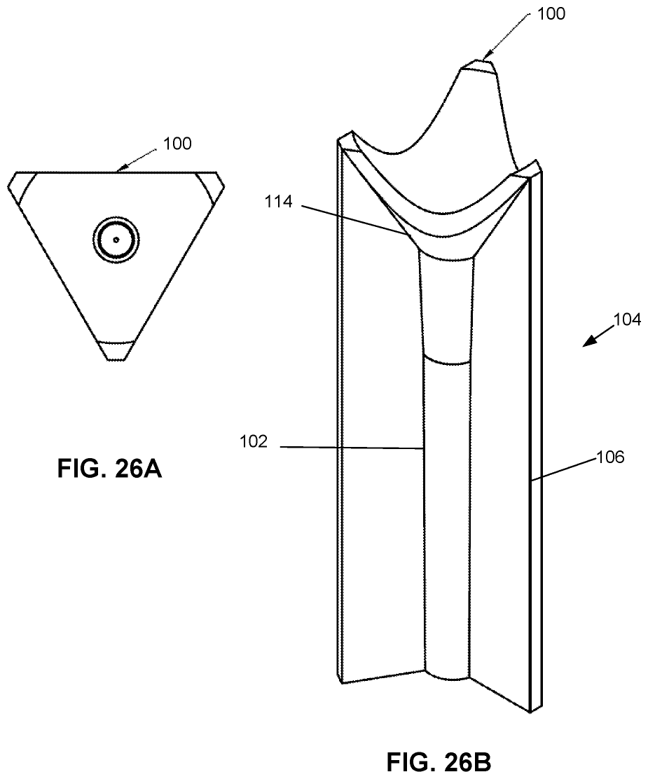

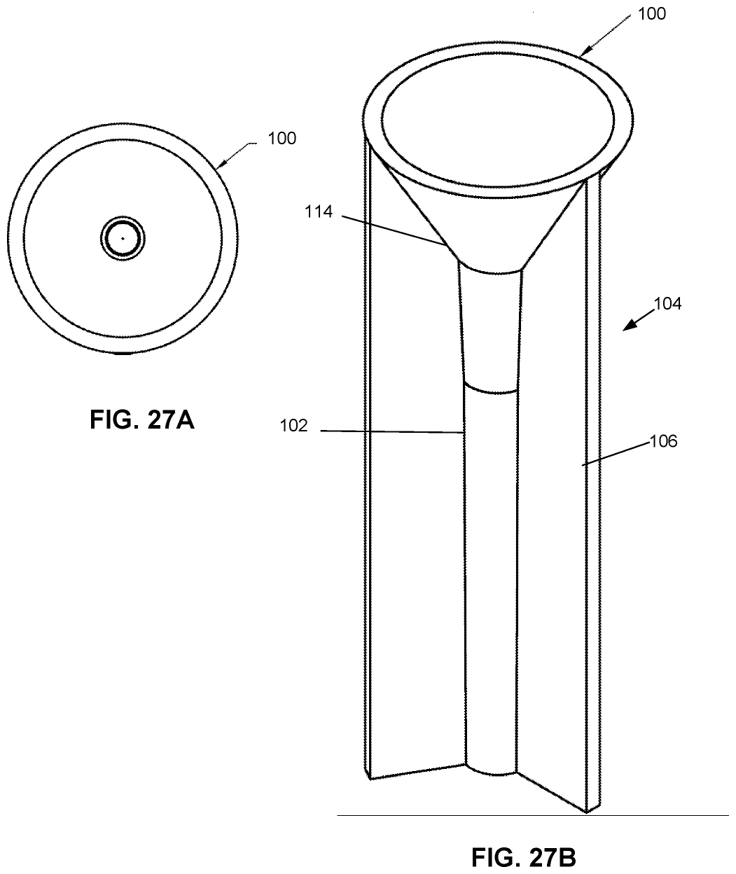

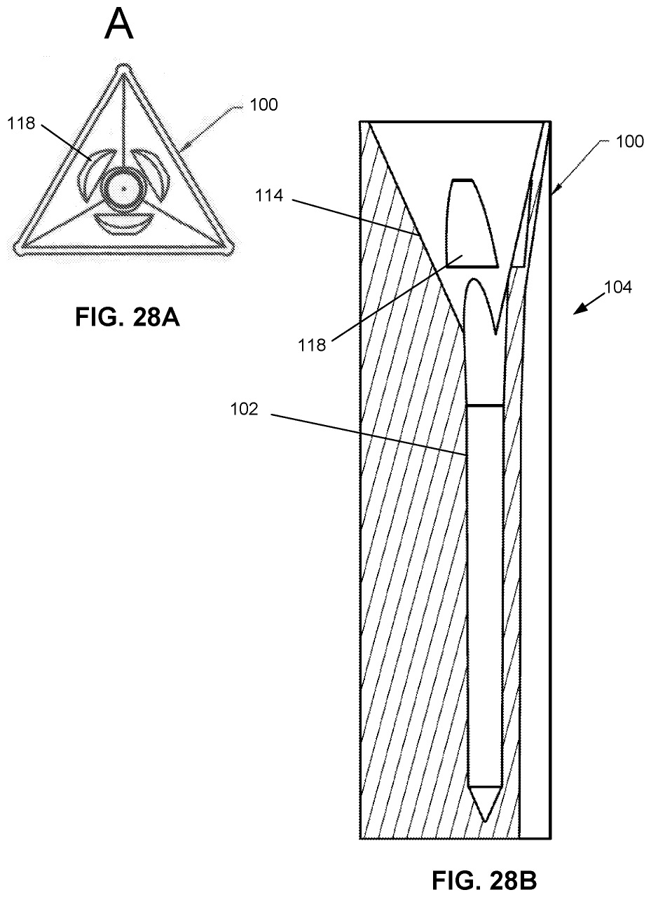

The proximal end of the cylindrical barrel may be tapered to have a larger opening. For example, the proximal end of the cylindrical barrel may further include a funnel 114, with a wide external facing open end that reduces in cross-section as it connects to the cylindrical barrel. The funnel has a wide opening that is operable to receive a hypodermic needle or a hypodermic needle sheath. The funnel may provide a sterile surface so that a needle tip has less chance of touching an unsterile surface when the hypodermic needle is being removed or replaced into the protective lumen. The funnel 114 may be either flat sided as shown in FIGS. 25A-25B, or rounded. In the case of a round-style funnel, the shape may be scalloped as seen in FIGS. 26A-26B or circular as shown in FIGS. 27A-27B. In additional examples, the interior of the funnel may be enlarged at the distal end so as to accommodate a Luer Lok hub. A cross-section and side view showing a Luer Lok hub 118 in the funnel is shown in FIGS. 28A-28B.

The funnel may assist in needle tip guidance for individuals experiencing tremor. Because this surface is part of the needle protective cover when it undergoes sterilization, touching the needle tip to the funnel during re-sheathing does not risk contamination.

The needle protective cover may be sterilized prior to use, such that it may be a single-use device. The needle protective cover may be packaged in a sterile condition, such that it is ready to use when the packaging is opened.

In some examples, the needle protective cover may be a replacement protective cover for hypodermic needles and would be placed on the needle by manufacturers prior to packaging and sterilization. In other examples, the needle protective cover may be friction fit on to existing needle manufacturer covers (not shown) after the user has removed the needle from its sterile packaging, but before the protective cover is removed from the needle the first time.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A needle protective cover comprising:
a cylindrical barrel having a proximal end and a distal end, the cylindrical barrel comprising a funnel at the proximal end, wherein the cylindrical barrel and funnel are operable to receive a hypodermic needle or hypodermic needle sheath; and
a support mechanism surrounding the cylindrical barrel and funnel, wherein the support mechanism is operable to prevent the needle protective cover from rolling on a surface and increases a distance from the funnel to the surface.

2. The needle protective cover of claim 1, wherein the support mechanism comprises a triangular body.

3. The needle protective cover of claim 1, wherein the support mechanism comprises one or more fins.

4. The needle protective cover of claim 3, wherein the support mechanism comprises two or more fins.

5. The needle protective cover of claim 4, wherein the fins are evenly spaced around the cylindrical barrel.

6. The needle protective cover of claim 4, wherein two or more fins are connected together to form a hollow triangular portion.

7. The needle protective cover of claim 4, wherein the distal end of the fins are flat, the distal end of the fins are extended beyond the cylindrical barrel and drawn to a point, or the cylindrical barrel is extended beyond the fins to a point.

8. The needle protective cover of claim 3, wherein the one or more fins comprise one or more openings.

9. The needle protective cover of claim 8, wherein the one or more openings are circular, oval, or rectangular.

10. The needle protective cover of claim 3, wherein the one or more fins have a flat edge, a pointed edge, a rounded edge, a scalloped edge, or serrated edge.

11. The needle protective cover of claim 1, further comprising a cylinder operable to hold a light emitter.

12. The needle protective cover of claim 11, wherein the cylinder is connected to and parallel to the cylindrical barrel or the cylinder is within the support mechanism.

13. The needle protective cover of claim 11, wherein the light emitter is a chemical light stick operable to emit in the visible light or UV portion of the spectrum.

14. The needle protective cover of claim 1, further comprising a visualization marker.

15. The needle protective cover of claim 14, wherein the visualization marker comprises metal or iodine loaded nanoparticles, a metal coil, and/or a pigment imbedded in the needle protective cover.

16. The needle protective cover of claim 1, wherein the proximal end of the funnel is flat, scalloped, or rounded.

17. The needle protective cover of claim 16, wherein the funnel further comprises a Luer Lock hub.

18. The needle protective cover of claim 1, wherein the needle protective cover is pre-sterilized prior to packaging.
